(19) United States
(12) Patent Application Publication
(10) Pub. No.: US 2014/0024046 A1
(43) Pub. Date: Jan. 23, 2014

(54) Exosomal Biomarkers for Cardiovasular Events

(71) Applicants: Dominique De Kleijn, Wijk bij Duurstede (NL); Gerard Pasterkamp, Austerlitz (NL); Leonardus Timmers, Utrecht (NL); Siu Kwan Sze, Singapore (SG)

(72) Inventors: Dominique De Kleijn, Wijk bij Duurstede (NL); Gerard Pasterkamp, Austerlitz (NL); Leonardus Timmers, Utrecht (NL); Siu Kwan Sze, Singapore (SG)

(73) Assignee: CAVADIS B.V., Utrecht (NL)

(21) Appl. No.: 13/995,760

(22) PCT Filed: Feb. 17, 2012
(86) PCT No.: PCT/EP12/00714

§ 371 Date: Oct. 1, 2013

(30) Foreign Application Priority Data
Feb. 18, 2011 (EP) EP/2011/052437
Mar. 24, 2011 (EP) EP2011/054567
Jul. 8, 2011 (EP) EP2011/061657

(51) Int. Cl. G01N 33/68

(52) U.S. Cl. CPC G01N 33/6893
USPC 435/7.21; 435/7.92; 530/350

(57) ABSTRACT

The present invention relates to a method of predicting the risk of a subject developing a cardiovascular event, comprising determining the presence of a biomarker that is indicative of the risk of developing a cardiovascular event in an exosome sample from the subject. The exosomes are suitably isolated from a body fluid selected from serum, plasma, blood, urine, amniotic fluid, malignant ascites, bronchoalveolar lavage fluid, synovial fluid, breast milk, saliva, in particular serum. Alternatively, the exosomes are present in a body fluid, in particular serum. The biomarker is selected from the proteins Vitronectin, Serpin F2, CD14, Cystatin C, Plasminogen, Nidogen 2, Serpin G1 or any combination of two or more of these proteins. The invention further relates to a method of diagnosing the occurrence of acute coronary syndrome in a subject, comprising determining the presence of a biomarker that is indicative of the occurrence of acute coronary syndrome in an exosome sample from the subject. In this method the biomarker is selected from Serpin F2, CD14, Cystatin C or combinations thereof.

### FIELD OF THE INVENTION

The invention relates to the field of risk stratification and/or patient stratification, more particular to the prognosis of risks on cardiovascular events such as stroke, transient ischemic attack (TIA) myocardial infarction (heart attack), cerebral bleeding and other major abnormalities occurring in the blood and to diagnosing acute ischemic coronary syndromes (ACS).

The invention relates in particular to a method of predicting the risk of a subject developing a cardiovascular event.

The present invention further relates to the diagnosis of acute ischemic coronary syndromes (ACS).

The invention also relates to kits and biomarkers for use in the methods.

### BACKGROUND OF THE INVENTION

Established cardiovascular risk factors, including dyslipidemia, smoking, hypertension and diabetes mellitus, have been incorporated into algorithms for cardiovascular risk assessment. However, the identification of patients who are at risk of developing cardiovascular disease remains difficult.

The identification of prognostic biomarkers would be of major added value in recognizing patients who are at risk of suffering future cardiovascular events and who could then be targeted for aggressive preventive measures. For primary cardiovascular events, the prognostic value of biomarkers is very limited since these biomarkers only moderately add to standard risk factors. For secondary events, prognostic biomarkers are non-existent.

The ideal approach in the search for biomarkers is an unbiased approach. Novel molecular techniques such as proteomics opened new possibilities for this purpose.

Recently in the laboratory of the present inventors, this technique was successfully used to discover biomarkers for cardiovascular disease in atherosclerotic plaques. Unfortunately, plaque material can only be obtained through invasive procedures. It is therefore a first object of the present invention to provide an alternative method for predicting the risk of a subject developing a cardiovascular event.

Patients with chest-pain are entering the emergency rooms of hospitals very frequently. Chest-pain, however, can result from many causes: gastric discomfort (e.g. indigestion), pulmonary distress, pulmonary embolism, dyspnea, musculoskeletal pain (pulled muscles, bruises) indigestion, pneumothorax, cardiac non-coronary conditions, and acute ischemic coronary syndrome (ACS).

Acute coronary syndrome (ACS) is usually one of three diseases involving the coronary arteries: ST elevation myocardial infarction (30%), non ST elevation myocardial infarction (25%), or unstable angina (38%). These types are named according to the appearance of the electrocardiogram (ECG/EKG) as non-ST segment elevation myocardial infarction (NSTEMI) and ST segment elevation myocardial infarction (STEMI). ACS is usually associated with coronary thrombosis.

The physician h to decide if the patient is having has a life threatening ischemic ACS or not. In the case of such an ischemic cardiac event, rapid treatment by opening up the occluded coronary artery is essential to prevent further loss of myocardial tissue.

Diagnosis of ACS is often not easy. For this, cardiac biomarkers have become an essential tool to define if a patient has a myocardial necrosis related to myocardial infarction. Favorable features of biomarkers of necrosis are high concentrations in the myocardium and absence in non-myocardial tissue, release into the blood within a convenient diagnostic time window and in proportion to the extent of myocardial injury, and quantification with reproducible, inexpensive, rapid, and easily applied assays (cited from ACC/AHA Guidelines, Circulation 116:803-877 (2007)).

The cardiac troponins possess many of these features and have gained wide acceptance as the biomarkers of choice. Myocardial necrosis now is defined by an elevation of troponin above the 99th percentile of normal.

Myocardial infarction, which is necrosis related to ischemia, is further defined by the addition of at least 1 of the following criteria: ischemic ST and T-wave changes, new left bundle-branch block, new Q waves, PCI-related marker elevation, or imaging showing a new loss of myocardium (cited from ACC/AHA Guidelines, Circulation 116:803-877 (2007)).

Although troponins can be detected in blood as early as 2 to 4 h after the onset of symptoms, elevation can be delayed for up to 8 to 12 h. This timing of elevation is similar to that of Creatine Kinase-MB but persists longer, for up to 5 to 14 days.

Accurate and rapid diagnosis of ACS based on a panel of biomarkers originating from different biological pathways is essential. Therefore, an urgent need exists for markers that can add to the diagnostic accuracy of Troponins. An earlier and more accurate detection than Troponins alone will result in a more rapid treatment with subsequent reduced loss of myocardial tissue.

It is thus a further object of the invention to provide the means for an accurate and rapid diagnosis of ACS.

### SUMMARY OF THE INVENTION

In the research that led to the invention, proteomic analyses were performed on human plaque and plasma samples. The procedure was hampered, however, by the presence of high-abundant plasma proteins such as albumin and immune-globulins, which complicated the detection of potentially interesting low-abundant proteins. Therefore sub-fractions of plasma were investigated for the presence of proteins that may have predictive value for cardiovascular events.

It was then found that protein constitution in plasma exosome samples from subjects that have suffered a cardiovascular event following the moment of sampling differs from that in patients who have not suffered such a cardiovascular event, and that this difference can be used for prognosis of patients.

Protein secretion out of the cells can occur directly after production (constitutive pathway) or is first stored in the cell and released after a trigger (regulatory pathway). Secretion, however, not only occurs with individual proteins but also occurs via vesicles containing a large number of proteins and RNA. These vesicles are formed with a selection of lipids, protein and RNA from the secreting cell and are released as an intact vesicle. Vesicles in the size of 50-100 nm are called exosomes and the release of exosomes has been described for various cell types, including reticulocytes, B- and T-lymphocytes, dendritic cells, mast cells, platelets, macrophages and alveolar lung cells. In several cell types, including T cells, platelets, dendritic cells and mast cells, secretion of exosomes is regulated by specific stimuli.

While early studies focused on their secretion from diverse cell types in vitro, exosomes have now been identified in body fluids such as urine, amniotic fluid, malignant ascites, broncho-alveolar lavage fluid, synovial fluid, breast milk, saliva and blood. Exosomes have a wide range of biological functions, including immune response, antigen presentation, intracellular communication and the transfer of RNA and proteins.

The present inventors found that since exosomes express an array of proteins that reflect the originating host cell, they contain valuable information regarding ongoing (patho)physiologic processes in the human body including information of future cardiovascular events.

This surprising finding now led to the present invention, which thus provides a method for predicting the risk of a cardiovascular event in a patient, based on the detection in plasma exosome samples and/or other micro-vesicles of smaller or larger size from said subject of proteins with prognostic value, herein after referred to as biomarkers or differentially present proteins.

The term ‘plasma exosome sample’ can refer both to a sample of isolated exosomes and a sample of body fluid, in particular serum or plasma, comprising exosomes.

According to the invention, in principle any biomarker with prognostic value may be used. In particular, however, specific markers were identified in or on plasma exosomes that have predictive value for secondary cardiovascular events.

In one embodiment, the invention thus provides a method of predicting the risk of a subject developing a cardiovascular event comprising detecting a biomarker in an exosome sample or other micro-vesicles of smaller or larger size from said subject, wherein said biomarker comprises at least one protein selected from the group of 6 proteins consisting of:

vitronectin (IPI:IPI00298971 SWISSPROT:VTNC_HUMAN,), Serpin F2 (IPI:IPI00879231, SWISSPROT:A2AP_HUMAN), CD14 (IPI:IPI00029260, SWISSPROT:CD14_HUMAN), Cystatin C (IPI:IPI00032293, SWISSPROT:CYTC_HUMAN), Plasminogen (IPI:IPI00019580,SWISSPROT:PLMN_HUMAN) Nidogen 2 (IPI:IPI00028908,SWISSPROT:NID2_HUMAN), SerpinG1 (IPI:IPI00291866; SWISSPROT: IC1_HUMAN).

According to the first aspect of the invention a biomarker comprises one protein or a set of multiple proteins. Such a biomarker is also identified herein as a profile or protein profile. A profile may comprise 1, 2, or more than 2 such as 3, 4, 5, 6 of the proteins: Vitronectin (IPI:IPI00298971 SWISSPROT:VTNC_HUMAN), Serpin F2 (IPI:IPI00879231, SWISSPROT:A2AP_HUMAN), CD14 (IPI:IPI00029260, SWISSPROT:CD14_HUMAN), Cystatin C (IPI:IPI00032293, SWISSPROT:CYTC_HUMAN), Plasminogen (IPI:IPI00019580, SWISSPROT:PLMN_HUMAN), Nidogen 2 (IPI:IPI00028908, SWISSPROT:NID2_HUMAN), SerpinG1 (IPI:IPI00291866; SWISSPROT: IC1_HUMAN). According to the invention a profile may be used that comprises any number and any combination of these proteins.

In a preferred embodiment of this aspect of the invention, the biomarker protein or a peptide fragment thereof is detected in exosomes or other vesicles somewhat larger or smaller in size that are preferably found in body fluids like serum, plasma or blood. Alternatively, exosomes or such other vesicles from other body fluids such as urine, amniotic fluid, malignant ascites, bronchoalveolar lavage fluid, synovial fluid, breast milk, saliva can be used.

In a further preferred embodiment, the biomarker protein or a peptide fragment thereof is detected in serum or plasma. In this embodiment, biomarkers that are attached to, anchored in or adhered to exosomes are detected.

According to the present invention, the cardiovascular event to be predicted is preferably selected from the following conditions: vascular death or sudden death, fatal or non fatal stroke, fatal or non fatal myocardial infarction, fatal or non fatal rupture of an abdominal aortic aneurysm, rupture of abdominal aortic aneurysm confirmed by laparatomy, vascular intervention, coronary artery disease, transient ischemic attack (TIA), peripheral arterial disease, acute coronary syndrome, heart failure or re-stenosis of carotid, coronary, femoral or other arteries.

The method of the present invention may suitably be used for risk stratification and/or patient selection (such as for clinical trials), for monitoring of disease, and the markers may be used as clinical biomarkers for safety and efficacy studies (e.g. as surrogate endpoint markers).

The invention also relates to a biomarker for use in the prognosis of the risk of a subject developing a cardiovascular event, comprising a protein selected from Vitronectin, Serpin F2, CD14, Cystatin C, Plasminogen, Nidogen 2, Serpin G1. In a further embodiment, the biomarker comprises a combination of two or more proteins selected from Vitronectin, Serpin F2, CD14, Cystatin C, Plasminogen, Nidogen 2, Serpin G1.

The cardiovascular event may be a primary event in a subject that has not yet suffered a cardiovascular event but is in particular a secondary event occurring in a subject already having suffered such an event before. According to the invention it is possible to discriminate between patients that already had a cardiovascular event and are at risk of suffering an additional event and patients who had such an event and do not have an increased risk of suffering a further event.

Suitably, the prognosis is made by using exosomes as the sample and preferably the biomarker comprising Vitronectin, Serpin F2, CD14, Cystatin C, Plasminogen, Nidogen 2, Serpin G1 or any combination thereof as the protein(s) to be detected.

In a further embodiment, the prognosis is made by using serum comprising the exosomes as the sample and preferably the biomarker comprising Vitronectin, Serpin F2, CD14, Cystatin C, Plasminogen, Nidogen 2 or any combination thereof as the protein(s) to be detected in or on the exosomes.

In ACS, the occlusion itself is often the result of a thrombotic event. This atherosclerotic plaque rupture brings the thrombogenic content of the plaque in contact with the blood initiating thrombosis and subsequent occlusion. Several leucocytes including platelets are involved in this sequence of events that, after activation, release microvesicles. Next to this, the ischemic event immediately activates endothelial cells that attract platelets that also become activated. This activation of endothelial cells and platelets is accompanied by the release of microvesicles that are secreted into the blood.

In the research leading to the second part of the invention it was contemplated that since secretion not only occurs with individual proteins like Troponin but also via vesicles containing a large number of proteins and RNA, the components found in, on or attached to the vesicles could also be used as markers.

These vesicles are formed with a selection of lipids, protein and RNA from the secreting cell and are released as an intact vesicle. They are generally called microvesicles and have a size between 20 and 1000 nm. From these microvesicles, exosomes are the best described particles having a size between 50 and 100 nm.

When an ACS occurs, microvesicles are secreted from several cells and tissues. The most obvious tissue is the myocardium. Apoptosis of cardiomyocytes occurs almost instantly after occluding the coronary artery and subsequent ischemia. The apoptopic cardiomyocytes secrete vesicles in the blood that are called apoptopic bodies.

In the research leading to this aspect of the present invention, the expression of particular proteins associated with microvesicles, in particular exosomes, were found to be suitable biomarkers to accurately diagnose ACS.

First, proteomic analyses were performed on human plasma samples. The procedure was hampered, however, by the presence of high-abundant plasma proteins such as albumin and immune-globulins, which complicated the detection of potentially interesting low-abundant proteins. Therefore sub-fractions of plasma were investigated for the presence of proteins that may have diagnostic value for the occurrence of an ACS.

It was then found that protein constitution in plasma exosome samples from subjects that had ACS following the moment of sampling differs from that in patients who did not have an ACS, and that this difference can be used for diagnosis of patients.

In the application the word “exosome” is thus intended to include other vesicles that are smaller than about 50 nm or larger than 100 nm but still fall within the range of about 20 to about 500 nm.

In several cell types, including T cells, platelets, dendritic cells and mast cells, secretion of exosomes is regulated by specific stimuli. While early studies focused on their secretion from diverse cell types in vitro, exosomes have now been identified in body fluids such as urine, amniotic fluid, malignant ascites, broncho-alveolar lavage fluid, synovial fluid, breast milk, saliva and blood. Exosomes have a wide range of biological functions, including immune response, antigen presentation, intracellular communication and the transfer of RNA and proteins.

The present invention shows that exosomes express an array of proteins that reflect the originating host cell and that they contain valuable information regarding ongoing (patho)physiologic processes in the human body including information on the occurrence of ACS.

This surprising finding now led to the aspect of the present invention that provides a method for the diagnosis of ACS in a patient, based on the detection of particular proteins in, on or attached to exosomes, which proteins are herein after referred to as biomarkers or differentially present proteins. The proteins can be detected either in or on or attached to isolated exosomes and in or on exosomes that are still present in a body fluid, in particular serum. According to the invention in principle any biomarker with diagnostic value may be used. In particular, however, specific markers were identified in/on plasma exosomes that have diagnostic value for ACS.

In one embodiment, the invention thus provides a method for the diagnosis of ACS comprising detecting a biomarker in an exosome sample or micro-vesicles of smaller or larger size from said subject, wherein said sample comprises at least one protein selected from the group of 3 proteins consisting of: Serpin F2 (IPI:IPI00879231, SWISSPROT:A2AP_HUMAN), CD14 (IPI:IPI00029260, SWISSPROT:CD14_HUMAN), Cystatin C (IPI:IPI00032293, SWISSPROT:CYTC_HUMAN).

The IPI numbers as disclosed herein between brackets refer to the International Protein Index (http://www.ebi.ac.uk/IPI), as indexed on Dec. 4, 2010 followed by Swissprot database Entry name as indexed on Nov. 30, 2010. The referenced index numbers (database accessions) as used herein include reference to fragments, isoforms and modifications thereof, hence the present invention foresees the use of fragments of the proteins as well as modifications and derivatives of the proteins disclosed herein as biomarkers in the context of the various aspects of the present invention.

According to the invention a biomarker comprises one protein or a set of multiple proteins. Such a biomarker is also identified herein as a profile or protein profile. According to this aspect of the invention a profile may comprise 1, 2 or 3 of the proteins Serpin F2 (IPI:IPI00879231, SWISSPROT:A2AP_HUMAN), CD14 (IPI:IPI00029260, SWISSPROT:CD14_HUMAN), Cystatin C (IPI:IPI00032293, SWISSPROT:CYTC_HUMAN) in any combination, in particular CD14 and Serpin F2 or Cystatin C and Serpin F2 or CD14 and Cystatin C or CD14, Serpin F2 and Cystatin C.

The skilled person will understand that instead of detecting the complete biomarker protein, one may detect peptide fragments of said biomarker proteins which are derived from the biomarker proteins by fragmentation thereof. The term peptide fragment as used herein refers to peptides having between 5 and 50 amino acids. These peptide fragments preferably provide a unique amino acid sequence of the protein, and are associated with the cardiovascular events as disclosed herein.

The proteins and/or peptide fragment may optionally be detected as chemically modified proteins and/or peptides, such chemical modification may for instance be selected from the group consisting of glycosylation, oxidation, (permanent) phosphorylation, reduction, myristylation, sulfation, acylation, acetylation, ADP-ribosylation, amidation, hydroxylation, iodination, and methylation. A large number of possible protein modifications is described in the RESID database at http://www.ebi.ac.uk/RESID (release Dec. 2, 2010) (Garavelli, J. S. (2004) The RESID Database of Protein Modifications as a resource and annotation tool; Proteomics 4: 1527-1533) and in Farriol-Mathis, N., Garavelli, J. S., Boeckmann, B., Duvaud, S., Gasteiger, E., Gateau, A., Veuthey, A., Bairoch, A. (2004) Annotation of post-translational modifications in the Swiss-Prot knowledge base. Proteomics 4(6): 1537-50. The skilled artisan is well aware of these modifications.

The biomarkers can be found in exosomes but also physically connected or linked to exosomes, which means both in or on their surface. When on their surface the biomarker can be either attached to the membrane, e.g. expressed on or in the membrane surface or anchored therein, or be in loose connection therewith, i.e. adhered to the exosome without being physically attached to or in the membrane. The biomarkers may also be part of the membrane. Of the biomarkers listed above Cystatin C is not attached to the membrane but rather adheres to it. CD14 is anchored in the membrane, and Serpin F2 has been associated with the membrane but it is unclear how it is attached.

It was found according to the invention that the biomarkers that are attached, anchored or adhered to the exosome can also be detected in samples of body fluid, in particular in serum.

In a preferred embodiment of the invention, the biomarker protein or a peptide fragment thereof is detected in, on or attached to exosomes that are preferably found in body fluids like serum, plasma or blood.

The invention further relates to a kit for performing any one of the methods disclosed herein, wherein the kit comprises means for detecting the presence of a biomarker as defined above. The means for detecting the presence of the biomarker are preferably antibodies, antibody fragments or antibody derivates or via mass spectrometry and flow cytometry. The antibody-based detection means optionally comprise a detectable label.

The kit of the invention is intended for use in a method of predicting the risk of a subject developing a cardiovascular disease by determining the presence of a biomarker in or on exosomes of the subject or for diagnosing ACS or for risk prediction for coronary heart disease in females. Thus, the kit may further comprise reagents and/or instructions for using the means for detecting a biomarker in any such method.

The present invention will be further illustrated in the Examples that follow and that are not intended to limit the invention in any way. Reference is made to the following figures:

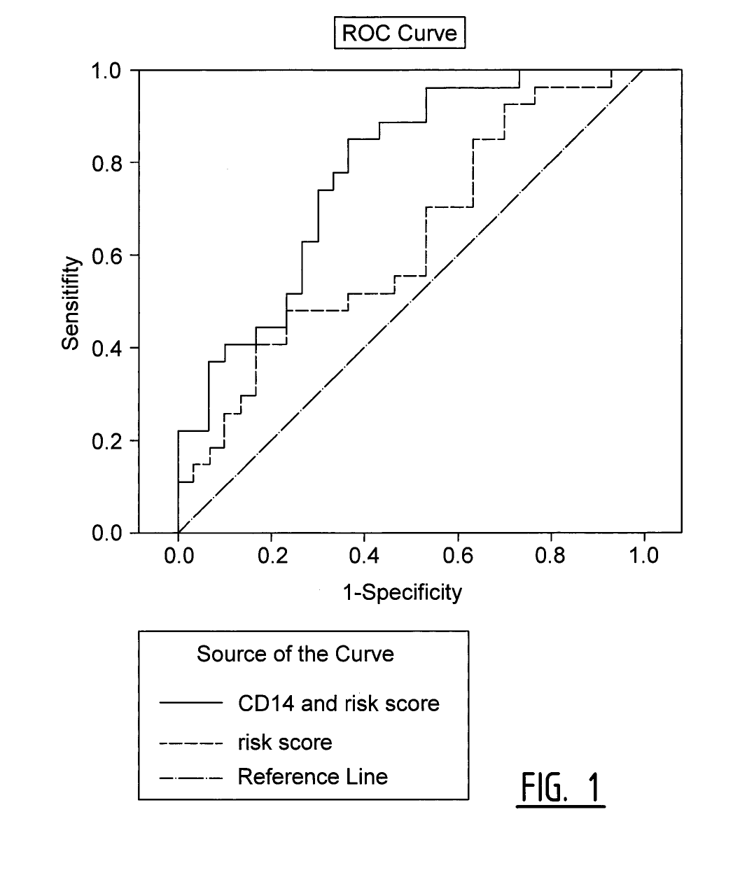

FIG. 1: the graph shows two ROC analyses for CD14. The solid black line is the ROC analysis for the CD14+risk factors (AUC=0.778, P=0.001); the broken black line is the ROC analysis for the risk factors alone (AUC=0.630, P 0.093). The solid grey line is the reference line and represents an AUC of 0.5 (that is, no discrimination).

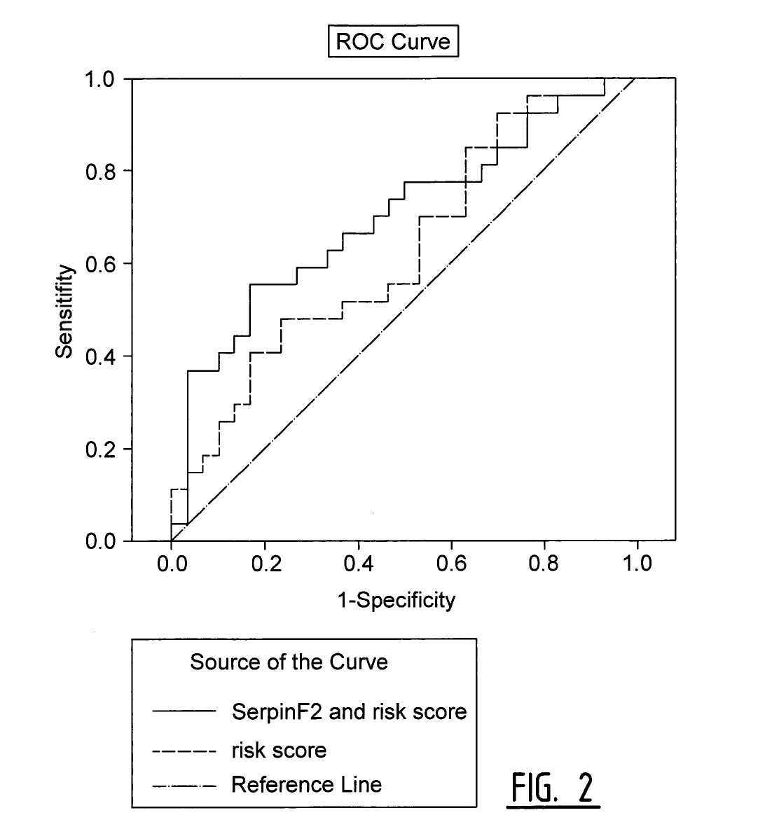

FIG. 2: the graph shows two ROC analyses for Serpin F2. The solid black line is the ROC analysis for the SerpinF2+risk factors (AUC=0.701, P=0.009); the broken black line is the ROC analysis for the risk factors alone (AUC=0.630, P=0.093). The solid grey line is the reference line and represents an AUC of 0.5 (that is, no discrimination).

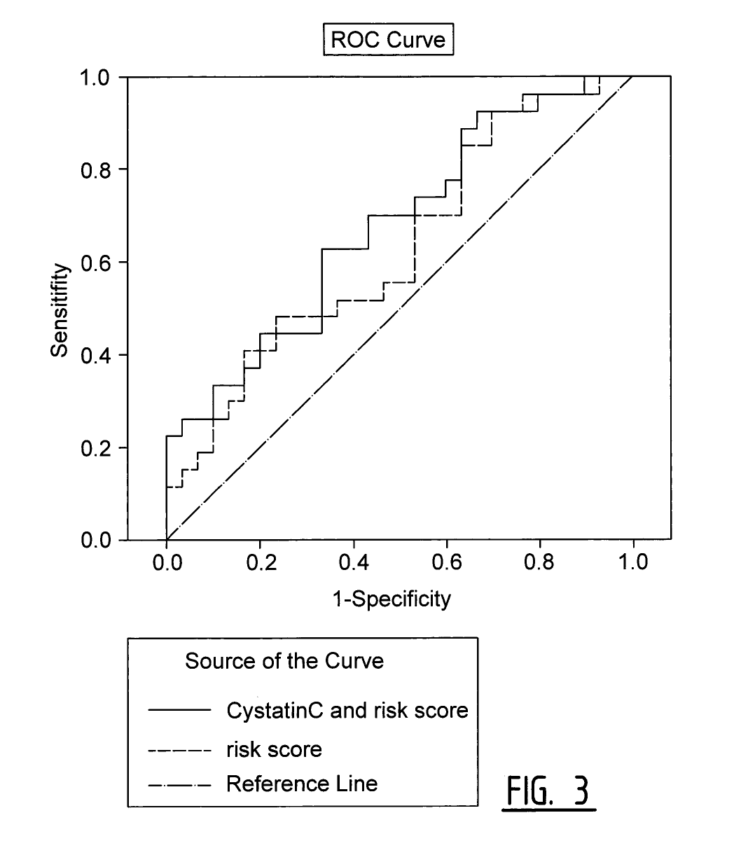

FIG. 3: the graph shows two ROC analyses for Cystatin C. The solid black line is the ROC analysis for the Cystatin C+risk factors (AUC=0.677, P=0.022); the broken black line is the ROC analysis for the risk factors alone (AUC=0.630, P=0.093). The solid grey line is the reference line and represents an AUC of 0.5 (that is, no discrimination).

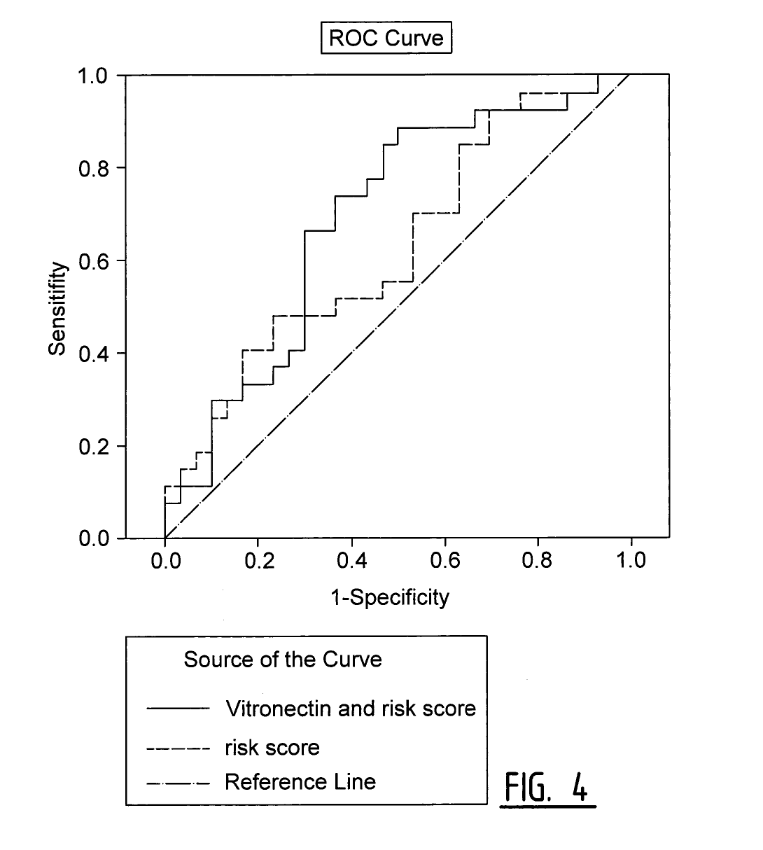

FIG. 4: the graph shows two ROC analyses for Vitronectin. The solid black line is the ROC analysis for the Vitronectin+risk factors (AUC=0.690, P=0.014); the broken black line is the ROC analysis for the risk factors alone (AUC=0.630, P=0.093). The solid grey line is the reference line and represents an AUC of 0.5 (that is, no discrimination).

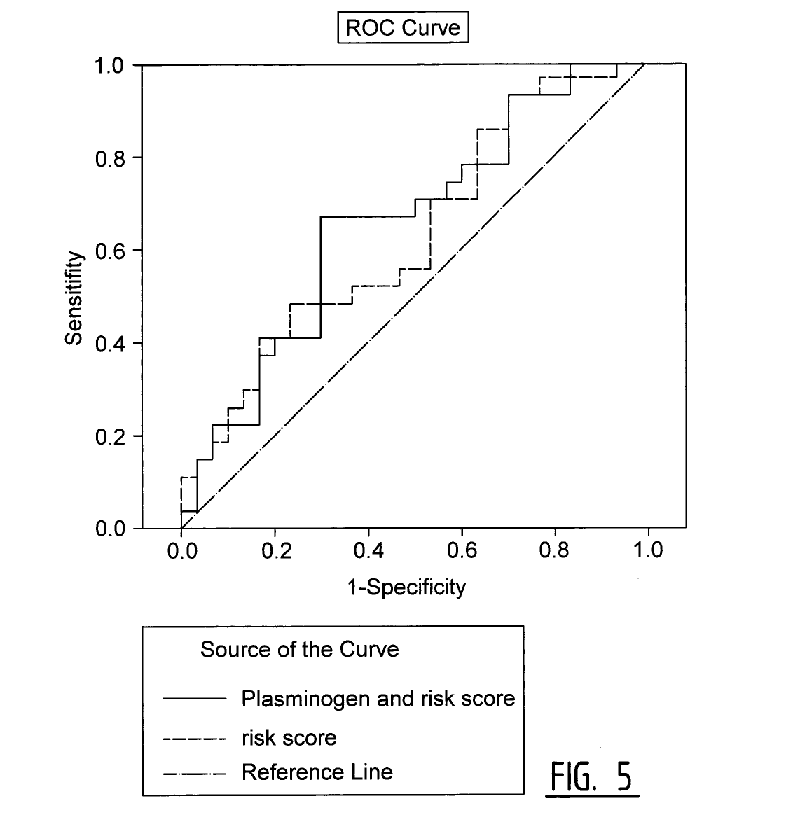

FIG. 5: the graph shows two ROC analyses for Plasminogen. The solid black line is the ROC analysis for the Plasminogen+risk factors (AUC=0.654, P=0.046); the broken black line is the ROC analysis for the risk factors alone (AUC=0.630, P=0.093). The solid grey line is the reference line and represents an AUC of 0.5 (that is, no discrimination).

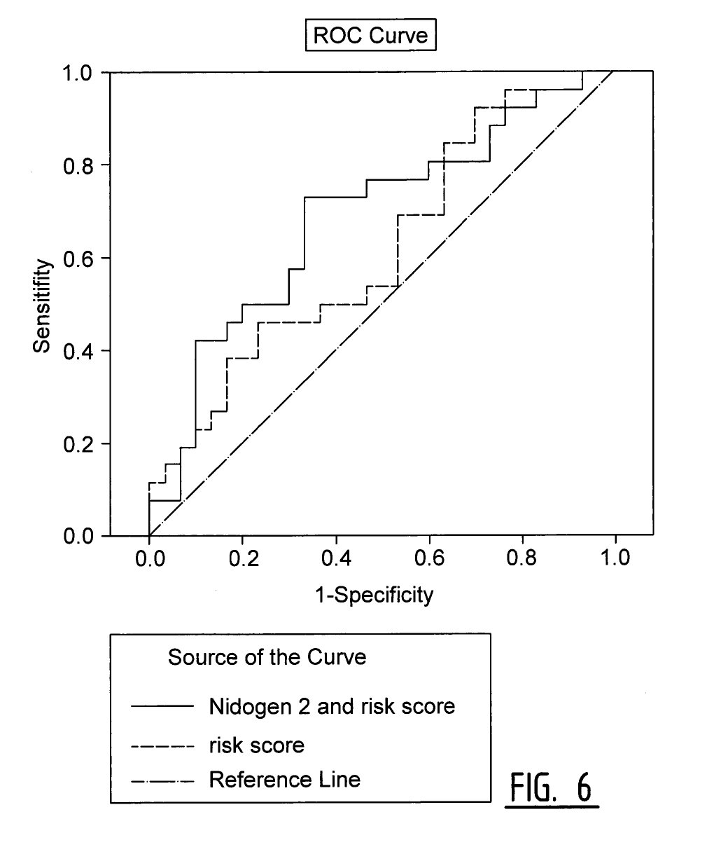

FIG. 6: the graph shows two ROC analyses for Nidogen 2. The solid black line is the ROC analysis for the Nidogen 2+risk factors (AUC=0.686, P=0.017); the broken black line is the ROC analysis for the risk re alone (AUC=0.630, P=0.093). The solid grey line is the reference line and represents an AUC of 0.5 (that is, no discrimination).

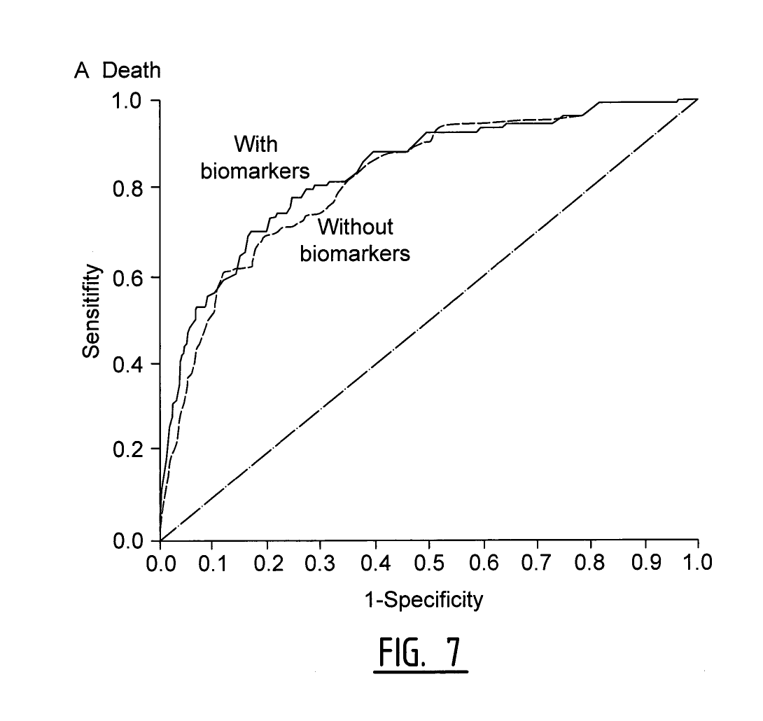

FIG. 7: a published ROC curve.

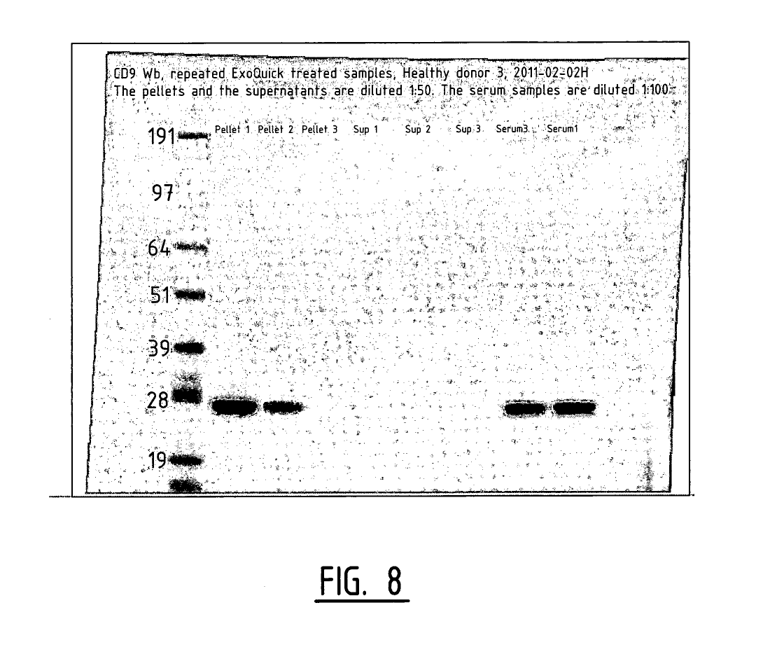

FIG. 8: Typical CD9 western blot showing CD9 levels in original serum (serum 3) and in exosome pellet after 1× (pellet 1) 2× (pellet 2) and 3× (pellet 3) exosome precipitation using Exoquick™. Serum 1 is loading control. Sup is Serum (Supernatant) after 1× (Sup 1), 2× (Sup 2) and 3× (Sup 3) precipitation.

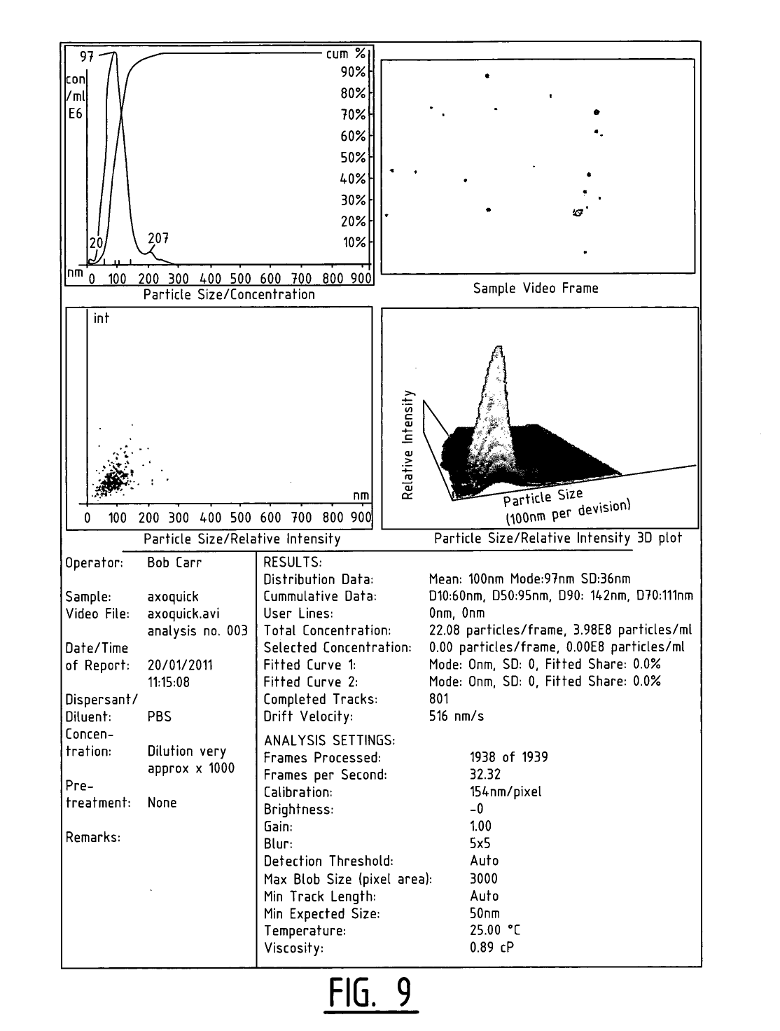

FIG. 9: Nanosight Sample Report of an exosome pellet after resuspension and dilution.

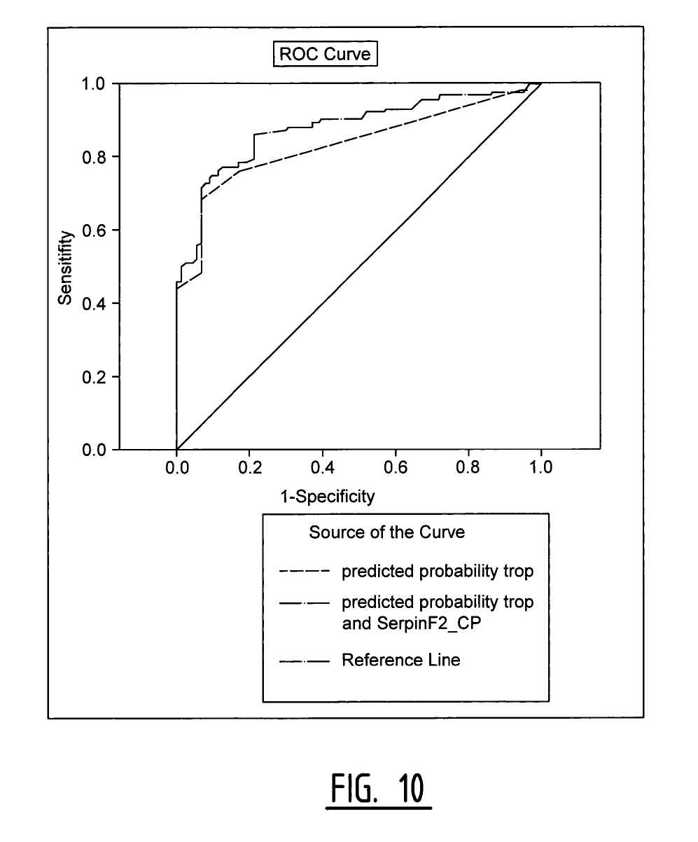

FIG. 10: Area under the curve analysis for Troponin (Trop, solid curve) measured in blood taken at intake of the patient with chest pain and Troponin plus Serpin F2 (SerpinF2_CP, dashed curve).

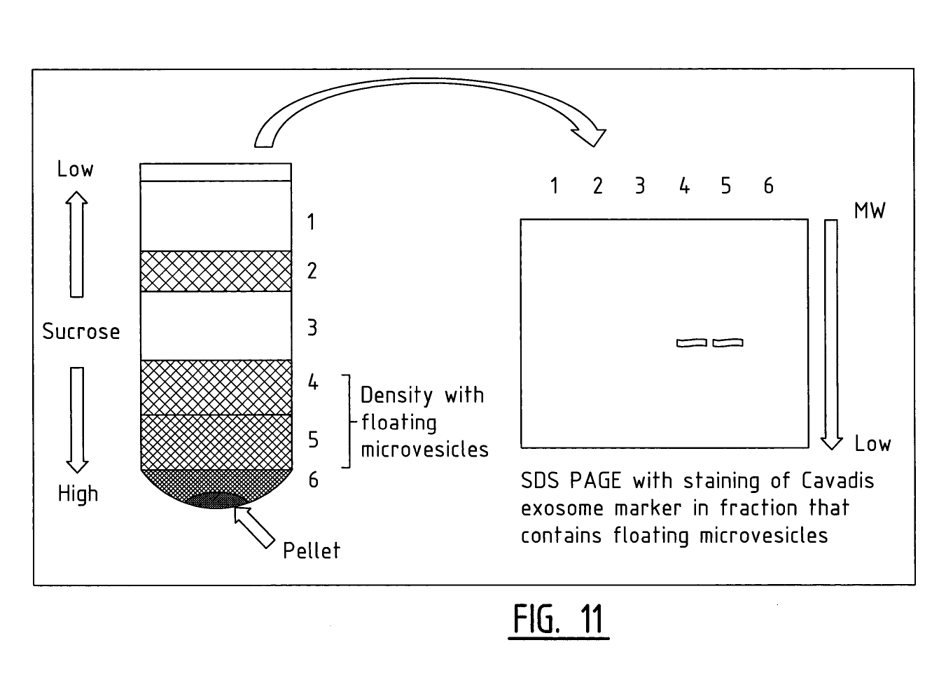

FIG. 11: Schematic representation of flotation experiment (left) and subsequent SDS PAGE for protein separation (right). Microvesicles preparation from plasma/serum samples was performed by ultracentrifugation on a linear sucrose gradient of 2.0-0.4 M. Microvesicles will float on a different sucrose gradient density based on their different buoyancy and density.

One subpopulation will be found in the lower density fraction while the other will be found in the higher density fraction. After ultracentrifugation, big aggregated proteins and debris ended up in the pellet at the bottom of the tube. All fractions were collected and subjected to SDS PAGE to separate all proteins on size prior to CD14, Cystatin C, Serpin F2, and Serpin G1 biomarker identification and quantification by Western Blot analysis.

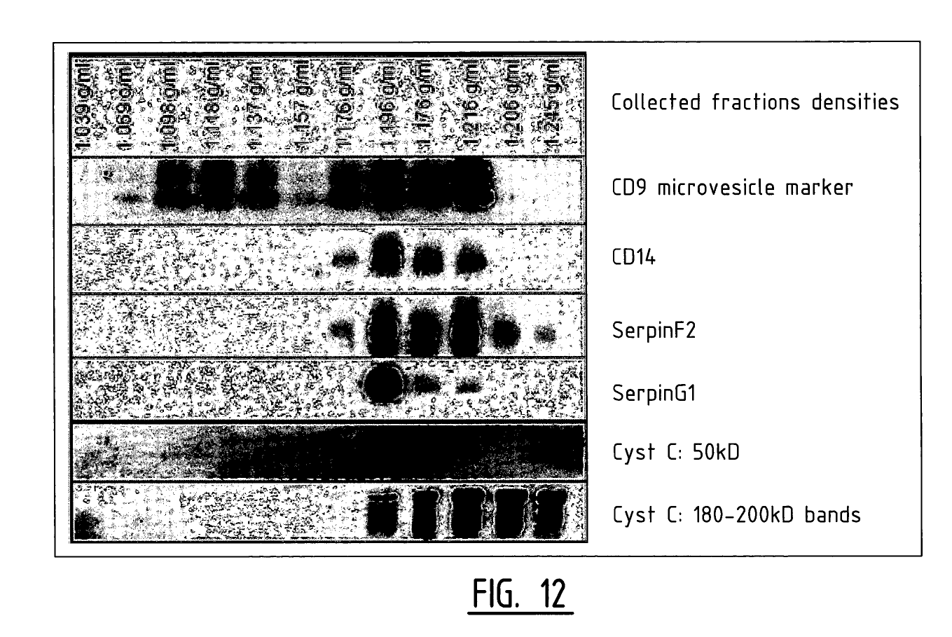

FIG. 12: Western Blot analysis on microvesicles obtained from the flotation experiment. CD9 protein was used as the microvesicle marker protein. All four biomarkers were found to be present in collected fractions of microvesicles: fractions densities of 1.176 g ml⁻¹ to 1.216 g ml⁻¹ for CD14; 1.196 g ml⁻¹ and 1.176 g ml⁻¹ for Cystatin C 50 kD and 1.196 g ml⁻¹ to 1.245 g ml⁻¹ for Cystatin C 180-200 kD; 1.176 g ml⁻¹ to 1.245 g ml⁻¹ for Serpin F2; and 1.196 g ml⁻¹ to 1.216 g ml⁻¹ for Serpin G1.

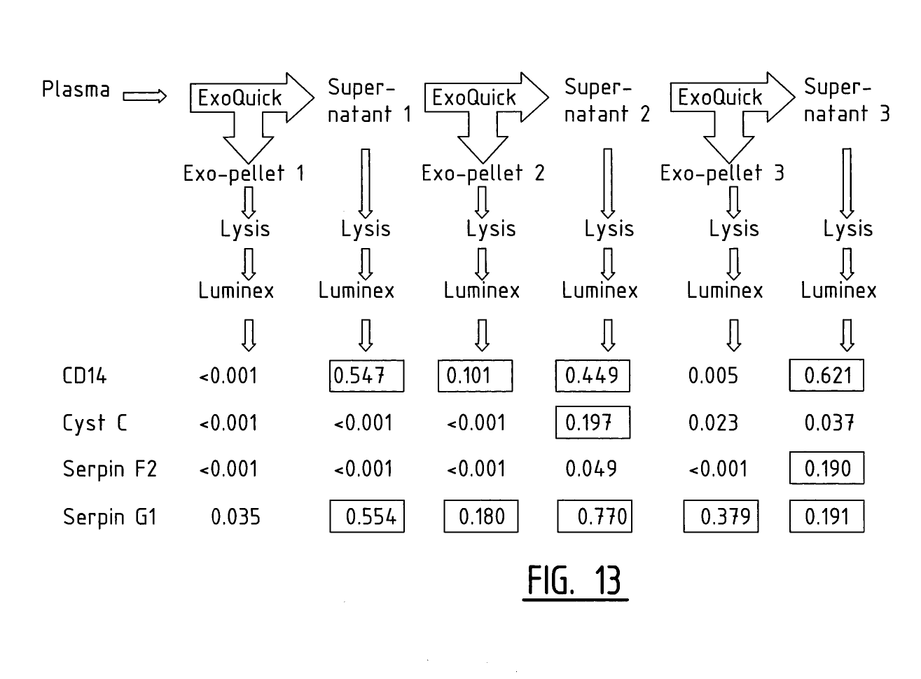

FIG. 13: Predictive biomarkers in microvesicles isolated with ExoQuick Precipitation Solution. Multiplex Luminex assay was used to validate the significant differences between events and controls of CD14, Cystatin C, Serpin F2, and Serpin G1 from patient plasma samples of 25 events vs 25 controls. P-values were determined using a Mann-Whitney test between events and controls using different sample input of either ExoQuick-pellet 1, 2, 3 or supernatant 1, 2, 3. CD14 and Serpin G1 lost their positive predictive values in supernatant 1. On the other hand, Cystatin C and Serpin F2 retained their positive predictive values in Exo-pellet 2 and Exo-pellet 3, respectively.

### EXAMPLES

### Example 1

### Quantitative Proteomics on Human Plasma Exosomes with Follow-Up

### Study Population and Design

The Athero-Express is a longitudinal vascular biobank study, which includes biomaterials from patients undergoing carotid and femoral end-arterectomy in two Dutch hospitals (UMC Utrecht and St. Antonius Hospital Nieuwegein). About 2000 patients have been included thus far. Plasma and tissue samples were obtained from all patients before (blood) or during end-arterectomy.

All patients underwent clinical follow-up 1 year after surgical intervention and filled in postal questionnaires 1, 2 and 3 years after the operation. When patients did not respond to the questionnaire, the general practitioner was contacted by phone. Adjudication of the outcome events was done by an independent outcome event committee that was blinded to laboratory results. Two members of the committee independently assessed all endpoints. In case of disagreement, a third opinion was obtained.

### The Exosomal Proteome

Plasma samples from 50 patients that suffered a coronary event during follow up and from 50 age and sex matched control patients, without a secondary event during follow up, were pooled separately and exosomes were isolated by filter separation and ultracentrifugation. Quantitative proteomics were used to identify the exosomal protein content, and allowed to compare the expression levels of the proteomes from patients that suffered events during follow up with the proteomes of control patients.

Exosomes were isolated from frozen human plasma by filter separation followed by ultracentrifugation (cf. Marie-Pierre Caby et al. Exosomal-like vesicles are present in human blood plasma; International Immunology, Vol. 17, No. 7, pp. 879-887).

Typical exosomal proteins such as CD9 and CD81 were detected in the exosome pellet using western blotting. FACS analysis with beads of defined sizes demonstrated that the pellet contains mostly particles of 50-100 nm which is in accordance with the size of exosomes.

### Protein Extraction and Digestion

The exosome pellets collected in the Athero-Express biobank plasma were after ultracentrifugation dissolved in 40 μl 6% SDS in HPLC pure water. Plaque protein was, after grinding the plaque material without any blood remains to powder, also extracted with 6% SDS. Digestion and subsequent labeling, HPLC separation and mass spectrometry analysis was identical for plaque and exosome proteins. The protein content was determined by 2-D Quant Kits. After protein reduction and alkylation, the protein mixture was diluted 20 times with 50 mM triethylammonium bicarbonate (TEAB) and protein digestion was initiated by adding trypsin in a 1:40 trypsin-to-protein ratio. The protein digests were desalted using a Sep-Pak C18 cartridge and dried in a Speedvac.

These digests were labeled with iTRAQ reagents according to the manufacturer's protocol. Briefly, digested proteins were reconstituted in 30 μl of dissociation buffer and mixed with 70 μl of ethanol-suspended iTRAQ reagents (one iTRAQ reporter tag per protein sample, mass tag 114-117 Dalton). Labeling reactions were carried out at RT for 1 hr before all the samples were mixed into a single tube and dried using a Speedvac.

### Strong Cation Exchange Fractionation

The combined iTRAQ labeled samples were reconstituted with 200 μl buffer A (10 mM KH₂PO₄, pH 3.0, 25% v/v acetonitrile), and loaded onto a PolySULFOETHYL A column (200 mm length×4.6 mm ID, 200-Å pore size, 5 μm particle size) on a Shimadzu prominence HPLC system. The sample was fractionated using a gradient of 100% buffer A for 5 min, 5-30% buffer B (10 mM KH₂PO₄, pH 3.0, 500 mM KCl and 25% v/v acetonitrile) for 40 min, 30-100% buffer B for 5 min, and finally 100% buffer B for 5 min, at a constant flow rate of 1 ml/min for a total of 60 min. The eluted fractions were monitored through a UV detector at 214 nm wavelength.

Fractions were collected at 1-min intervals and consecutive fractions with low peak intensity were combined. Finally, a total of 20 fractions was obtained and dried in a Speedvac. Each fraction was reconstituted in 0.1% trifluoroacetic acid and desalted. Desalted samples were dried in a Speedvac and stored at −20° C. prior to mass spectrometric analysis.

### Mass Spectrometric Analysis Using Q-STAR

The dried fraction was reconstituted in 100 μl of 0.1% formic acid. Each sample was analyzed two times using a Q-Star Elite mass spectrometer, coupled to an online Shimadzu prominence HPLC system. For each analysis, 50 μl of peptide mixture was injected and separated on a home-packed nanobored C18 column with a picofrit nanospray tip (75 μm ID×15 cm, 5 μm particles). The separation was performed at a flow rate of 20 μl/min with a splitter of a 90 min gradient. The mass spectrometer was set to perform data acquisition in the positive ion mode, with a selected mass range of 300-2000 m/z. Peptides with +2 to +4 charge states were selected for MS/MS and the time of summation of MS/MS events was set to 2 s. The three most abundantly charged peptides above a 5 count threshold were selected for MS/MS and dynamically excluded for 30 s with +30 mmu mass tolerance. Peptide quantification and protein identification were performed using ProteinPilot software v2.0.1 by searching the combined data from the 2 runs against the International Protein Index (IPI) human database (indexed Dec. 19, 2009). The Paragon algorithm in ProteinPilot software was used whereby trypsin was selected as the digestion agent and cysteine modification of methylethanethiosulfonate.

All proteins reported had an expectation value of less than 0.05 (unused score 1.3).

Quantitative proteomics were performed on exosomes from 50 patients that suffered a coronary event during follow up (Group 1) and from 50 matched control patients that did not suffer a secondary event during follow up (Group 2). Each group was run twice in the same iTraq experiment revealing data of 2 events proteomes and 2 control proteomes.

2148 different proteins were identified in the samples, including a large number of proteins identified earlier in exosome proteomics such as CD9, CD81, Annexins, Clathrin heavy chain, Enolase 1 and many more (Olver C, Vidal M. Proteomic analysis of secreted exosomes. Subcell Biochem. 2007; 43:99-131).

### Results of Exosome Proteomics

Group 1 and 2 were then compared using the quantitative iTRAQ data. Quantitative data were available from 2 pooled events samples (Group 1 in duplo) and 2 pooled control samples (Group 2 in duplo). Based on pilots, it was determined that a ratio of 1.2 and above means that there is significantly higher level of the protein in the event while a ratio of 0.8 and lower is a significant lower level in the event. First selection was based on proteins with identical duplo's (both below 0.8, both above 1.2 or both between 0.8 and 1.2).

Second selection was based on proteins with lower (events/controls<0.8) or higher (events/controls>1.2) expression in group 1 vs. group 2. This revealed a list of 116 proteins.

This list of 116 proteins was uploaded and analyzed in Ingenuity Pathway Analysis software (version 8.0). From the 116 proteins, 102 proteins were in the Ingenuity database. This revealed that the 102 proteins are different types of proteins, including transmembrane receptors, transporters and transcription regulators, proteins that are not present in plasma. Ingenuity analysis also showed that 3 canonical pathways are significantly overrepresented in these 102 proteins:

1. Acute Phase Response Signaling (p=3.5*10⁻¹¹)

2. Coagulation System (p=3.6*10⁻¹¹)

3. Atherosclerosis Signaling (p=3*10⁻¹)

Subsequently, this group of 102 differentially expressed proteins was complemented with a selection of plaque material derived proteins and finally narrowed down to a combined set of 34 selected exosome- and plaque-derived proteins for further validation in exosome samples of individual patient samples.

### Results of Plaque Protein Proteomics

Using the Athero-Express cohort, 40 carotid end-arterectomy patients were selected of which 20 had a secondary cardiovascular event during follow-up and 20 (age, sex and time to follow-up matched) controls that did not suffer from a secondary event during follow-up.

Quantitative proteomics was performed on plaque samples as for the exosome proteomics. However, since 40 individual plaques were analyzed, four plaque extracts were run simultaneously each differently labeled by the iTraq reagent (114, 115, 116, 117 resp.). Each run consisted of two plaque extracts of patients that suffered a cardiovascular event and for each patient a sex and age matched control, so in total four plaque extracts in two pairs of event and control.

After the search, an excel file was generated containing the protein ID and the relative value of the two event/control pairs for each of the protein IDs.

Analysis was performed after 10 runs including 20 pairs of events with matched controls with a total of 40 patients. Using Excel 2007 with the Merge Table Add-in, a total list of protein IDs was generated. Normalization between the different runs occurred via total peptide area correction.

Statistical analysis comparing events with controls using a Mann-Whitney test revealed 264 proteins that were significantly different (p<0.05) between events and controls in plaque.

### Selection of Exosome and Plaque-Derived Proteins

The plaque is the origin of atherosclerotic disease leading to cardiovascular events. For this, it is very likely that plaque proteins related to future cardiovascular events can also be found in, on, anchored or adhered to exosomes especially the plaque proteins that are related to the pathways over-represented in exosome proteins that differ between cardiovascular events and controls.

Having established that 3 canonical pathways (acute phase, coagulation and atherosclerosis) are over-represented in exosome proteins differentially expressed between events and controls, the 264 plaque-protein data-set with differentially expressed plaque proteins between events and controls was investigated in 2 ways.

Selection was based on the presence of proteins that are related to the 3 atherosclerosis related canonical pathways and for which 2 antibodies and a recombinant protein were available.

Also from the 112 exosome-derived proteins, markers were selected based on over-representation of 3 atherosclerosis related canonical pathways and the availability of 2 antibodies and a recombinant protein. From the selected plaque and exosome proteins for which antibodies and recombinant protein were available, 34 proteins were chosen for Luminex bead assay development. For 17 proteins out of those 34 proteins, a reproducible and quantitative Luminex bead assay was set up that could be used for measuring the protein content in connection to exosomes isolated from individual serum samples.

### Example 2

### Verification of the Selected Proteins in a Proof of Concept Study in Blood Samples of Individual Athero-Express Patients

### Study Objective

The objective of this study was to identify in blood samples of individual patients which of those 17 biomarkers were differentially expressed between patients suffering from a secondary coronary event and healthy controls.

### Study Design

Patients in this study underwent surgery of the carotid arteries because of a primary cerebral-vascular event i.e. a stroke or Transient Ischemic Attack (TIA) and were followed-up for three years. The 17 markers were measured in blood samples of patients who suffered from a secondary coronary event (29 samples) and age and sex matched controls (30 samples). The secondary coronary events were defined as myocardial infarction (fatal and non-fatal), cardiovascular death, sudden death, coronary angioplasty, and coronary artery bypass graft (CABG).

### Materials and Methods

Exosomes were isolated from the plasma using the ultracentrifugation technique. Proteins extracted from the exosome samples were measured in multiplex Luminex bead assays.

### Statistical Analyses

Statistical analyses were performed using the statistical software package PASW Statistics 17.0.2 (SPSS Inc, Chicago, Ill.). Discrimination (a measure of how well the model can separate events and controls) is most often measured by the area under the receiver operating characteristic (ROC) curve, an established method for assessing biomarkers (Hlatky et al. American Heart Association Expert Panel on Subclinical Atherosclerotic Diseases and Emerging Risk Factors and the Stroke Council. Criteria for evaluation of novel markers of cardiovascular risk: a scientific statement from the American Heart Association. Circulation. 2009 May 5; 119(17):2408-16).

ROC analyses were performed to determine the ability of the marker, in conjunction with a risk score, to distinguish between patients with and without coronary future events.

In an ROC analysis, the specificity and sensitivity of a specific test is given for different cut-off values of the test outcome. FIG. 7 depicts a published ROC curve. The ideal test, with optimal sensitivity and specificity will follow a curve with 1−specificity=0 (the y-axis) and then with sensitivity=1.0 (see bold line). A test without any value will follow the black straight line. The discriminative power of the test is provided as “area under the curve” (AUC). AUC values range between 0.5 (no discrimination) and 1.0 (perfect discrimination).

Statistical significance was set at P=0.05. The risk score was based on 7 traditional cardiovascular risk factors (gender, age, cholesterol, systolic blood pressure, smoking status, history of peripheral artery disease, and history of coronary artery disease).

### Results

When a new test for prediction of disease is evaluated it has to be compared with the risk predictors that are already available in the clinical arena. In this case these are the traditional risk factors (see above under the section “Statistical Analysis”). Therefore the AUC of the traditional risk factors alone were compared with the AUC of the traditional risk factors+the new biomarker. Thus an increase in AUC is explained by the new biomarker.

The AUC of the risk score alone was found to be 0.630 (P=0.093). Six of the 18 markers, assessed in conjunction with the risk score, showed an increase in the AUC: CD14 0.778 (P=0.001) (FIG. 1); Serpin F2 0.701 (P 0.009) (FIG. 2); Cystatin C 0.677 (P=0.022) (FIG. 3); Vitronectin 0.690 (P=0.014) (FIG. 4); Plasminogen 0.654 (P=0.046) (FIG. 5); and Nidogen 2 0.686 (P=0.017) (FIG. 6).

These six proteins are thus in particular useful as a biomarker in connection to exosomes as the sample to be tested in order to allow a reliable prognosis of a patient suffering a future cardiovascular event.

### Example 3

### Measurement of Biomarkers in Serum Samples without Exosome Isolation

### Study Objectives

The objective of the present example is to evaluate whether a favourable biomarker profile can rule out the chance of a cardiac event in the following years. For this the QICS study (Quick identification of acute chest pain patients study described in BMC Cardiovasc Disord. (2009) 9:24) was used.

In a cohort of carotid end-arterectomy patients (Athero-Express), exosome bound biomarkers predictive for secondary cardiovascular events were identified (Example 1 and 2). To assess if the predictive power for secondary cardiovascular events of Cystatin C, CD14 and Serpin F2 could be reproduced, serum of 240 patients of the QICS cohort was used to measure the expression of these three exosome-based biomarkers in serum samples with and without isolating the specific exosome fraction of the serum.

### Results

Measurement of Exosome-Based Markers in Serum Samples with Exosome Isolation

Cystatin C, Serpin F2 and CD14 were measured using Luminex multiplex technology on exosomes that were isolated with Exoquick™. Cystatin C (235 samples) was differentially expressed in patients that had a secondary coronary event and patients that did not have an event during follow-up (Mann-Whitney test, p-value: p<0.001). Serpin F2 (238 samples) with Mann Whitney test showed a p-value of p=0.008 between events and controls while CD14 gave a p-value of 0.126 (238 samples) showing comparable results as Example 1 and 2.

### Measurement of Exosome-Based Markers in Serum Samples Without Exosome Isolation

In order to investigate if the same three markers could also be measured in serum without exosome extraction, 200 QICS serum samples from the original 240 samples were used. Marker concentrations of Cystatin C, Serpin F2 and CD14 were directly measured in serum samples using the same Luminex multiplex as above. All three markers showed a significant difference between events and controls (p<0.001).

It was concluded that Cystatin C, CD14 and Serpin F2 can be measured in serum samples without exosome isolation as well as in isolated exosomes using Exoquick™ and that in both samples (with or without exosome isolation) these three markers are predictive for secondary coronary events.

### Cystatin C, CD14 and Serpin F2 are Connected to Exosomes

In order to prove that these markers are somehow connected to (i.e. in, on or associated with, attached to, anchored to, adhered to, etc.) exosomes, the exosomes were precipitated from serum and the relative decrease of the 3 markers in the serum measured. Four serum samples, each from different patients, were used. Serum concentrations of Serpin F2, Cystatin C and CD14 were measured before Exoquick™ induced exosome precipitation and after three consecutive cycles of Exoquick™ precipitation.

TABLE 1
Relative serum concentrations as a percentage of total
original serum levels of three biomarkers after three
consecutive cycles of Exoquick ™ extraction

| Marker | Serum | 1x Exoquick | 2x Exoquick | 3x Exoquick |
|---|---|---|---|---|
| Cystatin C | 100 | 92% +/− 10 | 68% +/− 8 | 42% +/− 9 |
| Serpin F2 | 100 | 75% +/− 8 | 46% +/− 21 | 9% +/− 8 |
| CD14 | 100 | 87% +/− 15 | 52% +/− 15 | 7% +/− 2 |

Table 1 shows that indeed the serum levels of markers progressively decrease, almost disappear after three consecutive Exoquick™ extraction cycles, thus proving the fact that these markers are exosome bound.

### Exoquick™ Isolates Exosomes Out of Serum

CD9 is a trans-membrane protein that is associated with the membrane of exosomes and is one of the most common exosome proteins and used as exosome marker. Again the same 4 serum samples were used as above. CD9 was measured by Western Blot analysis before Exoquick™ exosome precipitation and after 1, 2 and 3 times Exoquick™ precipitation.

Western blot analysis of the other 3 patient samples revealed the same results (FIG. 8). This shows that CD9 as a transmembrane protein can be measured in serum but that exosome precipitation by Exoquick™ removes the exosome marker CD9 out of the serum.

### Nanosight Exosome Visualization

Nanosight measures the Brownian movement of vesicles by shattering light on the exosome pellet re-suspended in PBS. The less the vesicles move the bigger they are. An exosome pellet after Exoquick™ precipitation was re-suspended and diluted at least a 100.000 time and brought on the Nanosight machine resulting in FIG. 9, which shows that the vesicles in the Exoquick™ pellet are a very homogenous population of vesicles with most vesicles between 36 and 142 nm and a small percentage (<5%) between 142 and 300 nm.

### Example 4

### The SMART Cohort

For validation of the exosomal biomarkers the SMART-MR Study was used, a cohort study within the Second Manifestations of ARTerial disease (SMART) Study, with the objective to investigate brain changes on MRI in patients with symptomatic atherosclerotic disease. Between May 2001 and December 2005, 1,309 patients newly referred to the University Medical Center Utrecht (UMCU) with manifest coronary artery disease, cerebral vascular disease, peripheral arterial disease or an abdominal aortic aneurysm, and without magnetic resonance (MR) contraindications were included.

During a single day visit to the UMCU, an MRI of the brain was performed, in addition to a physical examination, ultrasound of the carotid arteries, and blood and urine sampling. As part of the SMART study, risk factors, medical history, and functioning were assessed with questionnaires that the patients filled in before their visit to the medical center.

All cohort members were followed for clinical cardiovascular events, for a minimum of three years. From the 1309 patients, plasma was still available from 1060 patients for biomarker analysis. The SMART study and SMART-MR study were approved by the ethics committee of the UMCU and written informed consent was obtained from all participants.

### Area Under the Curve (AUC) Results of CystatinC, CD14, SerpinF2 and SerpinG1 for Cardiac Endpoints in a Received Operator Characteristics (ROC)

Discrimination (a measure of how well the model can separate between events and controls) is most often determined by receiver operating characteristics (ROC), an established method for assessing biomarkers. ROC analyses were performed to determine the ability of the marker, in conjunction with a risk score, to distinguish between patients with and without future coronary events.

Using a model based on traditional risk factors alone gave an AUC value of 0.65. Subsequently, each marker was assessed individually on top of the traditional risk factors followed by assessing combinations of markers on top of traditional risk factors. The highest AUC results of 0.68 were obtained using a model including the traditional risk factors plus three markers (CD14, SerpinF2 and SerpinG1) and a model including the traditional risk factors markers plus four markers (CD14, CystatinC, SerpinF2 and SerpinG1) (p=0.013 for the combination of three markers and p=0.028 for the combination of four markers based on the likelihood ratio test).

| | | Likelihood |
|---|---|---|
| | | ratio test to |
| | | compare model |
| | | fit against |
| Model | AUC [95% CI] | base model |
| Base model: Linear predictor or | 0.65 [0.58-0.73] | n/a |
| risk score based on risk factors | | |
| alone # | | |
| Base model & CD14 | 0.67 [0.60-0.75] | 0.018 |
| Base model & CyetatinC | 0.66 [0.58-0.73] | 0.331 |
| Base model & SerpinF2 | 0.66 [0.59-0.74] | 0.164 |
| Base model & SerpinG1 | 0.66 [0.59-0.73] | 0.201 |
| Base model & 3 markers (CD14, | 0.68 [0.61-0.76] | 0.013 |
| SerpinF2, SerpinG1 | | |
| Base model & 4 markers (CD14, | 0.68 [0.61-0.76] | 0.028 |
| SerpF2, Cystatin C, SerpG1) | | |

# Traditional risk factors are: age, smoking, diabetes, creatinine, BMI, total cholesterol to HDL ratio, CRP, antihypertensive medication, and a sum score of vascular history (cerebral, coronary, peripheral and AAA, where AAA counts for double)

The AUC reflects the overall added value of a model and does not directly indicate its clinical value, therefore the Net Reclassification Index of the biomarkers were assessed.

### Net Reclassification Index

The Net Reclassification Index (NRI) is a tool to assess what effect a biomarker will have in classifying patients in pre-specified risk groups. The NRI analysis is a relatively new phenomenon in statistics. The American Heart Association recently published a new set of criteria for the evaluation of novel cardiovascular biomarkers, which included the NRI method. (Hlatky et al. American Heart Association Expert Panel on Subclinical Atherosclerotic Diseases and Emerging Risk Factors and the Stroke Council. Criteria for evaluation of novel markers of cardiovascular risk: a scientific statement from the American Heart Association published in Circulation 2009 May 5; 119(17):2408-16).

In Table 2 below the NRI for the exosome markers CystatinC, CD14, SerpinF2 and SerpinG1 are depicted.

TABLE 2

| Aggregate NRI: | | | SMART PREDICTION MODEL WITH EXOSOME PANEL | | |
|---|---|---|---|---|---|
| 15.5% shifting power | | | PANEL = CystatinC, CD14, SerpinF2, SerpinG1 | | |
| SMART (N = 1060) | | | <5% | 5-10% | >10% |
| SMART | <5% | Total N = 527 | 451 | 74 | 2 |
| PREDICTION | | Reclassified (%) | 85.6% | 14.0% | 0.4% |
| MODEL | | Observed Risk (%) | 4% | 8% | 0% |
| TRADITIONAL | 5-10% | Total N = 389 | 86 | 279 | 24 |
| RISK | | Reclassified (%) | 22.1% | 71.7% | 6.2% |
| FACTORS | | Observed Risk (%) | 2% | 5% | 21% |
| | >10% | Total N = 136 | 0 | 34 | 102 |
| | | Reclassified (%) | 0.0% | 25.0% | 75.0% |
| | | Observed Risk (%) | 0% | 3% | 19% |

On the vertical axis, categories of patients are plotted that were divided in risk groups (<5% risk, 5-10% risk and >10% risk on a cardiac event during follow up) based on a model using only traditional risk factors. Patients were divided in 3 groups: Group 1 (<5% risk) consisted of 527 patients (451+74+2), Group 2 (5-10% risk) consisted of 389 patients (86+279+24). Group 3 (>10% risk) consisted of 136 patients (102+34).

The patients were then reclassified by adding the exosome markers to the prediction model. It is evident that patients were shifted to a higher risk category (for instance 74 patients were shifted from the category <5% towards 5-10% risk category) while other patients were shifted to a lower category. The key question remains: did this reclassification based on the exosome markers result in better prediction? As next step the number of patients that were correctly and incorrectly shifted to other risk categories was calculated. Calculations learned that the aggregated percentages of high and low reclassification led to a Net Reclassification Index of 15.5% which is a much better risk classification than based on traditional risk factors alone.

Take for instance the group of patients categorized by traditional risk factors in the group 5-10%. Now 86 patients were reclassified in a lower risk group. These patients indeed had an event rate of 2%, indicating that this group was correctly shifted to a lower risk category. On the other hand 24 patients were reclassified in a higher risk group. In this group an event rate of 21% was observed, indicating that this group of patients indeed suffered from a higher risk for a secondary cardiovascular event.

In this model SerpinG1 is an important biomarker for the exosome panel. As shown in Table 3 below, each of the markers individually achieves on top of the traditional risk factors a reclassification effect ranging from 6.0% for CystatinC to 9.8% for SerpinF2, and for CD14 and SerpinG1 at 8.0% and 8.4%, respectively. These percentages already demonstrate the impact on patient reclassification of the individual markers. The combinations of 4 markers on top of the traditional risk factors, however, yields the best and clinically very relevant NRI score of 15.5%.

TABLE 3

| Model | NRI |
|---|---|
| Base model: Linear predictor or risk score based | NA |
| on risk factors alone# | |
| Base model & CD14 | 8.0% |
| Base model & CyatatinC | 6.0% |
| Base model & SerpinF2 | 9.8% |
| Base model & SerpinG1 | 8.4% |
| Base model & 4 markers | 15.5% |

#risk factors are: age, smoking, diabetes, creatinine, BMI, total cholesterol to hdl ratio, CRP, antihypertensive medication, and a sumscore of vascular history (cerebral, coronary, peripheral and AAA, where AAA counts for double)

### Example 5

### Quantitative Proteomics on Human Plasma Exosomes with Follow-Up

### Study Population and Design

The Athero-Express is a longitudinal vascular biobank study, which includes biomaterials from patients undergoing carotid and femoral end-arterectomy in two Dutch hospitals (UMC Utrecht and St. Antonius Hospital Nieuwegein). About 2000 patients have been included thus far. Plasma and tissue samples were obtained from all patients before (blood) or during end-arterectomy.

All patients underwent clinical follow-up 1 year after surgical intervention and filled in postal questionnaires 1, 2 and 3 years after the operation. When patients did not respond to the questionnaire, the general practitioner was contacted by phone. Adjudication of the outcome events was done by an independent outcome event committee that was blinded to laboratory results. Two members of the committee independently assessed all endpoints. In case of disagreement, a third opinion was obtained.

### The Exosomal Proteome

Plasma samples from 50 patients that suffered an ACS during follow up and from 50 age and sex matched control patients, without any secondary event during follow up, were pooled separately and exosomes were isolated by ultracentrifugation. Quantitative proteomics were performed on the exosomal protein content, and allowed to compare the expression levels of the proteomes from patients that suffered events during follow up with the proteomes of control patients.

Exosomes were isolated from frozen human plasma by filter separation followed by ultracentrifugation.

Typical exosomal proteins such as CD9 and CD81 were detected in the exosome pellet using western blotting. FACS analysis with beads of defined sizes demonstrated that the pellet contains mostly particles of 50-100 nm which is in accordance with the size of exosomes.

### Protein Extraction and Digestion

The exosome pellets collected in the Athero-Express biobank plasma were after ultracentrifugation dissolved in 40 μl 6% SDS in HPLC pure water. Plaque protein was, after grinding the plaque material without any blood remains to powder, also extracted with 6% SDS. Digestion and subsequent labeling, HPLC separation and mass spectrometry analysis was identical for plaque and exosome proteins. The protein content was determined by 2-D Quant Kits. After protein reduction and alkylation, the protein mixture was diluted 20 times with 50 mM triethylammonium bicarbonate (TEAB) and protein digestion was initiated by adding trypsin in a 1:40 trypsin-to-protein ratio. The protein digests were desalted using a Sep-Pak C18 cartridge and dried in a Speedvac.

These digests were labeled with iTRAQ reagents according to the manufacturer's protocol. Briefly, digested proteins were reconstituted in 30 μl of dissociation buffer and mixed with 70 μl of ethanol-suspended iTRAQ reagents (one iTRAQ reporter tag per protein sample, mass tag 114-117 Dalton). Labeling reactions were carried out at RT for 1 hr before all the samples were mixed into a single tube and dried using a Speedvac.

### Strong Cation Exchange Fractionation

The combined iTRAQ labeled samples were reconstituted with 200 μl buffer A (10 mM KH₂PO₄, pH 3.0, 25% v/v acetonitrile), and loaded onto a PolySULFOETHYL A column (200 mm length×4.6 mm ID, 200-A pore size, 5 μm particle size) on a Shimadzu prominence HPLC system. The sample was fractionated using a gradient of 100% buffer A for 5 min, 5-30% buffer B (10 mM KH₂PO₄, pH 3.0, 500 mM KCl and 25% v/v acetonitrile) for 40 min, 30-100% buffer B for 5 min, and finally 100% buffer B for 5 min, at a constant flow rate of 1 ml/min for a total of 60 min. The eluted fractions were monitored through a UV detector at 214 nm wavelength.

Fractions were collected at 1-min intervals and consecutive fractions with low peak intensity were combined. Finally, a total of 20 fractions were obtained and dried in a Speedvac. Each fraction was reconstituted in 0.1% trifluoroacetic acid and desalted. Desalted samples were dried in a Speedvac and stored at −20° C. prior to mass spectrometric analysis.

### Mass Spectrometric Analysis Using Q-STAR

The dried fraction was re-constituted in 100 μl of 0.1% formic acid. Each sample was analyzed two times using a Q-Star Elite mass spectrometer, coupled to an online Shimadzu prominence HPLC system. For each analysis, 50 μl of peptide mixture was injected and separated on a home-packed nanobored C18 column with a picofrit nanospray tip (75 μm ID×15 cm, 5 μm particles). The separation was performed at a flow rate of 20 μl/min with a splitter of a 90 min gradient.

The mass spectrometer was set to perform data acquisition in the positive ion mode, with a selected mass range of 300-2000 m/z. Peptides with +2 to +4 charge states were selected for MS/MS and the time of summation of MS/MS events was set to 2 s. The three most abundantly charged peptides above a 5 count threshold were selected for MS/MS and dynamically excluded for 30 s with +30 mmu mass tolerance.

Peptide quantification and protein identification were performed using ProteinPilot software v2.0.1 by searching the combined data from the 2 runs against the International Protein Index (IPI) human database (indexed Dec. 19, 2009). The Paragon algorithm in ProteinPilot software was used whereby trypsin was selected as the digestion agent and cysteine modification of methylethanethiosulfonate.

All proteins reported had an expectation value of less than 0.05 (unused score 1.3).

Quantitative proteomics were performed on exosomes from 50 patients that suffered an ACS during follow up (Group 1) and from 50 matched control patients that did not suffer a secondary event during follow up (Group 2). Each group was run twice in the same iTraq experiment revealing data of 2 events proteomes and 2 control proteomes.

2148 different proteins were identified in the samples, including a large number of proteins identified earlier in exosome proteomics such as CD9, CD81, Annexins, Clathrin heavy chain, Enolase 1 and many more (Olver C, Vidal M. Proteomic analysis of secreted exosomes. Subcell Biochem. 43:99-131 (2007)).

### Results of Exosome Proteomics

Group 1 and 2 were then compared using the quantitative iTRAQ data. Quantitative data were available from 2 pooled events samples (Group 1 in duplo) and 2 pooled control samples (Group 2 in duplo). Based on pilots, it was determined that a ratio of 1.2 and above means that there is significantly higher level of the protein in the event while a ratio of 0.8 and lower is a significant lower level in the event. First selection was based on proteins with identical duplo's (both below 0.8, both above 1.2 or both between 0.8 and 1.2).

Second selection was based on proteins with lower (events/controls<0.8) or higher (events/controls>1.2) expression in group 1 vs. group 2. This revealed a list of 116 proteins.

This list of 116 proteins was uploaded and analyzed in Ingenuity Pathway Analysis software (version 8.0). From the 116 proteins, 102 proteins were in the Ingenuity database. This revealed that the 102 proteins are different types of proteins, including transmembrane receptors, transporters and transcription regulators, proteins that are not present in plasma. Ingenuity analysis also showed that 3 canonical pathways are significantly overrepresented in these 102 proteins:

Acute Phase Response Signaling (p=3.5*10-11)
Coagulation System (p=3.6*10-11)
Atherosclerosis Signaling (p=3*10⁻⁴)

Subsequently, this group of 102 differentially expressed proteins was complemented with a selection of plaque material derived proteins and finally narrowed down to a combined set of 34 selected exosome and plaque derived proteins for further validation in exosome samples of individual patient samples.

### Results of Plaque Protein Proteomics

Using the Athero-Express cohort, 40 carotid end-arterectomy patients were selected of which 20 had a secondary cardiovascular event during follow-up and 20 (age, sex and time to follow-up matched) controls that did not suffer from a secondary event during follow-up.

Quantitative proteomics was performed on plaque samples as for the exosome proteomics. However, since 40 individual plaques were analyzed, four plaque extracts were run simultaneously each differently labeled by the iTraq reagent (114, 115, 116, 117 resp.). Each run consisted of two plaque extracts of patients that suffered a cardiovascular event and for each patient a sex and age matched control, so in total four plaque extracts in two pairs of event and control.

After the search, an excel file was generated containing the protein ID and the relative value of the two event/control pairs for each of the protein IDs.

Analysis was performed after 10 runs including 20 pairs of events with matched controls with a total of 40 patients. Using Excel 2007 with the Merge Table Add-in, a total list of protein IDs was generated. Normalization between the different runs occurred via total peptide area correction.

Statistical analysis comparing events with controls using a Mann-Whitney test revealed 264 proteins that were significantly different (p<0.05) between events and controls in plaque.

### Selection of Exosome and Plaque-Derived Proteins

The plaque is the origin of atherosclerotic disease leading to cardiovascular events. For this, it is very likely that plaque proteins related to future cardiovascular events can also be found in exosomes especially the plaque proteins that are related to the pathways over-represented in exosome proteins that differ between patients suffering from a cardiovascular events and healthy controls. Having established that 3 canonical pathways (acute phase, coagulation and atherosclerosis) are over-represented in exosomes, the 264 protein data-set with differentially expressed plaque proteins between events and controls was investigated in 2 ways.

Selection was based on the presence of proteins that are related to the 3 atherosclerosis related canonical pathways and for which 2 antibodies and a recombinant protein were available.

Also from the 112 exosome-derived proteins, markers were selected based on over-representation of 3 atherosclerosis related canonical pathways and the availability of 2 antibodies and a recombinant protein.

From the selected plaque and exosome proteins for which antibodies and recombinant protein were available, 34 proteins were chosen for Luminex bead assay development. For 17 proteins out of those 34 proteins (including Cystatin C, Serpin F2 and CD14), a reproducible and quantitative Luminex bead assay was set up that could be used for measuring the protein content in exosomes isolated from individual serum samples.

### Example 6

### Verification of a Selection of Differentially Expressed Proteins in a Proof of Concept Study in Blood Samples of Individual Patients (QICS Study)

### The Quick Identification of Acute Chest Pain Study (QICS)

### Study Objective

One of the objectives of the QICS study is the identification of sensitive predictive markers in acute chest pain patients. We will test, presentational symptoms, traditional risk factors, individual biomarkers, a profile of biomarkers, coronary calcium score, coronary stenosis/plaque volume. Biomarkers will be retrospectively tested after defrosting of deep frozen blood taken on presentation.

### Methods and Design

The Quick Identification of acute Chest pain Study (QICS) will investigate whether a combined use of specific symptoms and signs, electrocardiography, routine and new laboratory measures, adjunctive imaging including electron beam (EBT) computed tomography (CT) and contrast multi-slice CT (MSCT) will have a high diagnostic yield for patients with acute chest pain.

All patients are investigated according a standardized protocol in the Emergency Department. Serum and plasma are frozen for future analysis for a wide range of biomarkers at a later time point. The final diagnosis non cardiac chest pain, unstable angina, non ST elevation myocardial infarction, and ST elevation myocardial infarction, with registration of troponin, short term outcome, and long term outcome for secondary coronary events is recorded.

### Materials and Methods

### Isolation and Measurement

Cystatin C, Serpin F2 and CD14 were measured using Luminex multiplex technology on/in or attached to exosomes that were isolated with Exoquick™ from 250 ul of serum of individual QICS patients.

### Statistical Analyses

Statistical analyses were performed using the statistical software package PASW Statistics 17.0.2 (SPSS Inc, Chicago, Ill.). Discrimination (a measure of how well the model can separate events and controls) is most often measured by the area under the receiver operating characteristic (ROC) curve, an established method for assessing biomarkers (Hlatky et al. American Heart Association Expert Panel on Subclinical Atherosclerotic Diseases and Emerging Risk Factors and the Stroke Council. Criteria for evaluation of novel markers of cardiovascular risk: a scientific statement from the American Heart Association. Circulation 119(17):2408-16 (2009)).

ROC analyses were performed to determine the ability of the marker, in conjunction with a risk score, to distinguish between patients with chestpain without an acute coronary syndrome and patients with chestpain that do have an acute coronary syndrome.

### Results

Cystatin C (235 samples) was differentially expressed in patients that had an ACS and patients that did not have an ACS when entering the emergency room with acute chest pain p-value: p=0.003). Serpin F2 (238 samples) showed a p-value of p<0.001 between ACS and non-ACS while CD14 gave a p-value of 0.002 (238 samples)

The strongest marker Serpin F2 was analyzed to see if it had additional value in diagnosing acute coronary syndrome on top of Troponin levels measured at the intake of the patient.

ROC curves (FIG. 10) show that the area under the curve increases from 0.835 for Troponin alone to 0.881 for Troponin plus Serpin F2. Serpin F2 plus the maximum levels of troponin measured (Tropmax) is significantly different from Tropmax alone and Serpin F2 plus High sensitive (Hs)-Troponin is also significantly different from Hs-Troponin alone showing the added value of Serpin F2.

### Area Under the Curve

| | | | | Asymptotic 95% | |
|---|---|---|---|---|---|
| | | | | Confidence | |
| | | | | Interval | |
| Test Result | | Std. | Asymptotic | Lower | Upper |
| Variable(s) | Area | Error^{a} | Sig.^{b} | Bound | Bound |
| predicted | .835 | .027 | .000 | .783 | .888 |
| probability trop | | | | | |
| predicted | .881 | .023 | .000 | .837 | .925 |
| probability trop | | | | | |
| and Serpin F2_CP | | | | | |

The test result variable(s): predicted probability trop has at least one tie between the positive actual state group and the negative actual state group.
^{a}Under the nonparametric assumption
^{b}Null hypothesis: true area = 0.5

SerpinF2_CP and 3 varsions of troponin

| | | |
|---|---|---|
| SerpinF2_CP & troponin | 0.881 [0.837-0.925] | <0.001 |
| SerpinF2_CP & tropmax | 0.994 [0.988-1.000] | <0.001 |
| SerpinF2_CP & Hs-Troponin | 0.965 [0.943-0.987] | 0.011 |

### Example 7

### Verification and Validation of Predictive Exosome-Based Biomarkers Isolated from Microvesicles

### Study Objective

Important biological component such as membrane-linked protein markers are often present in microvesicles found in body fluids such as urine, amniotic fluid, malignant ascites, bronchoalveolar lavage fluid, synovial fluid, breast milk, and saliva. The objective of the experiments was to validate the presence of four protein biomarkers of CD14, Cystatin C, Serpin F2, and Serpin G in isolated microvesicles from plasma/serum through flotation experiment, Western Blot, and ExoQuick precipitation as described in the material and method section.

For this purpose, sucrose gradient centrifugation and ExoQuick isolation were used to isolate microvesicles from plasma samples.

Sucrose gradient separation using ultra-centrifugation on plasma samples is based on the different buoyancy (density) of microvesicles compared to free protein and large protein complexes. In sucrose gradient centrifugation, large complexes will go to the bottom of the tube (highest density) while free proteins will go to the top of the gradient (lower density). Microvesicles will float in the intermediate density layers that are also identified by microvesicle markers like CD9.

ExoQuick solution manufactured by System Biosciences (SBI) was used to precipitate microvesicles/exosomes out of the plasma samples according to the manufacturer's protocol

### Materials and Method

### Sucrose Gradient Separation Method

### 1. Sample Preparation

Citrate-anticoagulated human whole blood samples were collected and centrifuged at 1850×g for 10 minutes at room-temperature to eliminate cell debris. The resulting clear solution on the very top layer known as blood citrate plasma was transferred into a 15 ml tube, snapped freeze in liquid nitrogen, and stored in −80° C. for further use.

### 2. Sample Isolation

All plasma samples were thawed before use at room temperature. 2 ml plasma was taken out and centrifuged at 2000×g for 30 minutes at room temperature. The supernatant was transferred into a new tube and diluted in 1:1 ratio with 1×PBS for an ultracentrifugation run at 10000×g for 30 minutes at 4° C. After this first ultracentrifugation run, the supernatant was pipetted into a new tube for the second ultracentrifugation run at 110000×g for 1 hour at 4° C.

When necessary, PBS was added to equalize the volume in each tube prior to any of the centrifugation steps. After the second run, the supernatant was aspirated using an aspirator leaving the pellet containing microvesicles/exosomes undisturbed at the bottom of the tube. The pellet was resuspended in 20 μl 1×PBS, and mixed with 1.5 ml of 2.5 M sucrose in a new ultracentrifuge tube.

The suspension was carefully overlaid with decreasing molarities of sucrose of 700 μl each; 2.000 M; 1.886 M; 1.771M; 1.657 M; 1.543 M; 1.429 M; 1.314 M; 1.200 M; 1.086 M; 0.971M; 0.857 M; 0.743 M; 0.629 M; 0.514 M; 0.400 M prior to an overnight (or at least for 15 hours) ultra-centrifugation run at 200000×g at 4° C. with a low acceleration and braking.

Approximately 12 different fractions of 1 ml each were harvested the next day from 1 tube. Each of 1 ml fraction was mixed thoroughly, 900 μl per fraction was transferred into a new tube, and diluted with 0, 1% BSA in 1×PBS for another ultracentrifugation at 110000×g at 4° C. After one hour of centrifugation, the supernatant and all excess liquid was carefully decanted using an aspirator. The pellet containing microvesicles/exosomes or large protein complexes was resuspended in 55 μl of SDS sample buffer and directly used for Western Blot analysis.

### Western Blot Analysis

### 1. Sample Preparation and Analysis

The well established Western Blot procedure transfers proteins after separation on size to a carrier, and was used to analyze the presence of proteins in the fraction samples obtained from the previous sucrose gradient separation method. 10 μl of the sample and 7 μl of marker (SeeBlue®Plus2 Prestained Standard (1×)) was loaded onto a 4-12% Bis-Tris gel The gel was run at 200 V for 50 minutes in 1×MOPS buffer. After running, the gel was removed from its casting tray and soaked in transfer buffer prior to the proteins transfer from the gel onto the carrier; the PVDF membrane. Transfer from the gel to the PVDF membrane was done at 100 V for 1 hour. The membrane was blocked in a blocking solution containing 5% milk in PBS with 0.1% Tween for 1 hour at room temperature.

A working concentration of primary antibodies of CD9 (0.5 μg/ml), CD14 (2.5 μg/ml), Cystatin C (2.5 μg/ml), Serpin F2 (2.5 μg/ml), and Serpin G1 (2.5 μg/ml) were separately prepared and diluted in a total volume of 4 ml of 1×PBS containing 51 milk and 0.11 Tween. Each of them was added into their respective membrane and followed by an overnight incubation at 4° C. The membrane was washed 3×5 minutes with 1×PBS containing 0.1% Tween prior to incubation for 1 hour at room temperature with a secondary antibody labeled with HRPO. The membranes were washed 3×5 minutes with 1×PBS containing 0.1% Tween and 1×5 minutes with 1×PBS only. The Electro-chemiluminescent (ECL) substrate was equally spread on the membrane and incubated for 5 minutes at room temperature in the dark before image analysis using the Image Lab software was carried out.

### ExoQuick Precipitation Method

### 1. Exosome Isolation

The plasma samples were centrifuged at 3000×g for 15 minutes at room temperature prior to use. A pre-treated 0.45 μm filter with 100 μl of pre-heated MQ water at 37° C. was prepared, centrifuged at 10000×g for 2 minutes at room temperature, and transferred into a new empty filter tube. The plasma was added into the pre-treated filter followed by centrifugation at 12000×g for 10 minutes at room temperature. 250 μl of filtered plasma was taken and mixed thoroughly with 63 μl of ExoQuick solution for an overnight incubation at 4° C. The next morning, the precipitated exosomes were collected as a pellet using centrifugation at 1500×g for 30 minutes at room temperature. After removing off the supernatant, the pellet was centrifuged again at 1500×g for 5 minutes at room temperature to remove the remaining supernatant.

### 2. Protein Isolation

The pellet was resuspended in Roche Complete Lysis-M buffer containing protease inhibitors (EDTA-free) and incubated for 30 minutes at room temperature. To lyse a pellet derived from 250 μl of plasma, 100 μl of Roche Complete Lysis-M buffer was used. To help with the lysis, the pellet was pipetted up and down for a couple of times and continued to incubate for another 10 minutes at room temperature. A pre-treated 0.22 μm filter with 50 μl of Roche Complete Lysis-M buffer was prepared, centrifuged at 1000×g for 2 minutes at room temperature, and transferred into a new empty filter tube. The suspension was added to this pre-treated filter followed by centrifugation at 15000×g for 10 minutes at room temperature. The solution was collected after filtration and stored in 20 μl aliquots at −80° C.

### Results

Identification and Characterization of Biomarkers Isolated from Microvesicles

Microvesicles containing protein markers were separated, via ultracentrifugation on a continuous sucrose gradient from large aggregates which sedimented in the pellet as shown in FIG. 11. After centrifugation the different layers were taken out from the centrifugation tube and (after density measurement) used for Western Blot analysis. A panel of antibodies directed against CD14, Cystatin C, Serpin F2, and Serpin G1 was used in Western Blot analysis depicted in FIG. 12. CD9 protein is used as the vesicle protein marker since it is one of the most abundant protein families found in the membrane of microvesicles.

CD14 signal was detected in the densities fractions comprised between 1.176 g ml⁻¹ and 1.216 g ml⁻¹. Floating vesicles containing Serpin F2 and Serpin G1 were also found in densities fractions ranging from 1.176 g ml⁻¹ to 1.245 g ml⁻¹ and 1.196 g ml⁻¹ to 1.216 g ml⁻¹, respectively. Furthermore, two forms of Cystatin C differed on their molecular weight were visualized in the analysis. The 50 kD Cystatin C were found in the collected densities fractions of 1.196 g ml⁻¹ and 1.176 g ml⁻¹ whereas Cystatin C of 180-200 kD was indicated in densities fractions from 1.196 g ml⁻¹ to 1.245 g ml⁻¹. The results confirmed successful isolation of microvesicles containing CD14 Cystatin C, Serpin F2, and Serpin G1 via flotation experiment and verified by Western Blot analysis. The occurrence of each marker in more than one density fraction is likely due to different subpopulation of plasma membrane vesicles.

Validation of Predictive Biomarkers from Circulating Exosomes Isolated with ExoQuick

Microvesicles were isolated by overnight incubation of plasma samples obtained from 25 patients suffered a secondary coronary event during follow-up and 25 controls that did not have an event during follow-up with ExoQuick Precipitation Solution, resulting in a pellet at the bottom of the tube. This microvesicle pellet was lysed with Roche lysis-M buffer and used for CD14, Cystatin C, Serpin F2, and Serpin G1 Luminex detection and quantification. The measurement of these four markers was performed using both the pellet and the supernatant.

The exosome precipitation procedure was repeated three times as illustrated in FIG. 13. All four biomarkers showed a significant difference (p<0.05) between patients with a secondary cardiovascular event and controls. However, for CD14 and Serpin G1 the significant difference between events and controls was lost in supernatant 1 while for Cystatin C the significant difference between events and controls was no longer detected in supernatant 2. Unlike the rest, Serpin F2 maintained its significant difference between events and controls in supernatant 1, Exo-pellet 2, supernatant 2, and Exo-pellet 3 but not in supernatant 3 as indicated in FIG. 13.

Hence, this study shows that significant discrimination between events and controls for the CD14, Cystatin C, Serpin F2, and Serpin G1 levels is only present in the lysed microvesicles after the first ExoQuick precipitation.

1. A method of predicting the risk of a subject developing a cardiovascular event, comprising determining the presence of a biomarker that is indicative of the risk of developing a cardiovascular event in an exosome sample from the subject.

2. The method as claimed in claim 1, wherein the biomarker is selected from Vitronectin, Serpin F2, CD14, Cystatin C, Plasminogen, Nidogen 2, Serpin G1.

3. The method as claimed in claim 2, wherein the biomarker is any combination of two or more proteins selected from Vitronectin, Serpin F2, CD14, Cystatin C, Plasminogen, Nidogen 2, Serpin G1.

4. The method as claimed in claim 1, wherein the cardiovascular event is selected from vascular death or sudden death, fatal or non fatal stroke, fatal or non fatal myocardial infarction, fatal or non fatal rupture of an abdominal aortic aneurysm, rupture of abdominal aortic aneurysm confirmed by laparatomy, vascular intervention, coronary artery disease, transient ischemic attack (TIA), peripheral artherial disease, acute coronary syndrome, heart failure or restenosis of carotid, coronary, femoral or other arteries.

5. A method of diagnosing the occurrence of acute coronary syndrome in a subject, comprising determining the presence of a biomarker that is indicative of the occurrence of acute coronary syndrome in an exosome sample from the subject.

6. The method as claimed in claim 5, wherein the biomarker is selected from Serpin F2, CD14, Cystatin C.

7. The method as claimed in claim 6, wherein the biomarker is any combination of two or more proteins selected from Serpin F2, CD14, Cystatin C.

8. The method as claimed in claim 1, wherein the exosome sample consists of exosomes that are isolated from a body fluid selected from serum, plasma, blood, urine, amniotic fluid, malignant ascites, bronchoalveolar lavage fluid, synovial fluid, breast milk, saliva, in particular serum.

9. A kit comprising a detector configured to detect the presence of a biomarker selected from the group consisting of Serpin F2, CD14, Cystatin C and combinations thereof.

10. The kit as claimed in claim 11, wherein the detector comprises antibodies, antibody fragments or antibody derivatives, optionally comprising a detectable label.

11. The kit as claimed in claim 9, further comprising at least one of reagents and instructions for using the detector in a method of.

12. A biomarker for use in the prognosis of the risk of a subject developing a cardiovascular event, comprising a protein selected from Vitronectin, SerpinF2, CD14, Cystatin C, Plasminogen, Nidogen 2, Serpin G1.

13. The biomarker as claimed in claim 12, wherein the biomarker comprises a combination of two or more proteins selected from Vitronectin, SerpinF2, CD14, Cystatin C, Plasminogen, Nidogen 2, Serpin G1.

14. The biomarker as claimed in claim 12, wherein for the prognosis of the risk of a subject developing a cardiovascular event the biomarker is detected in an exosome sample of the subject.

15. The biomarker as claimed in claim 14, wherein the exosome sample consists of isolated exosomes.

16. The biomarker as claimed in claim 14, wherein the exosome sample is a sample of a body fluid that comprises exosomes and is in particular serum.
